(19) United States
(12) Patent Application Publication
(10) Pub. No.: US 2006/0270625 A1
(43) Pub. Date: Nov. 30, 2006

(54) Nutraceuticals for the treatment of neuropathy

(75) Inventors: Aaron I. Vinik, Norfolk, VA (US); Jorge L. Jacot, Virginia Beach, VA (US)

Correspondence Address: WILMER CUTLER PICKERING HALE AND DORR LLP, 1875 PENNSYLVANIA AVE., NW, WASHINGTON, DC, 20004, US

(73) Assignee: Eastern Virginia Medical School, Norfolk, VA (US)

(21) Appl. No.: 11/431,830

(22) Filed: May 11, 2006

Related U.S. Application Data
(60) Provisional application No. 60/680,521, filed on May 13, 2005.

(51) Int. Cl. A61K 31/714; A61K 31/375; A61K 31/4415; A61K 31/455; A61K 31/525; A61K 31/51; A61K 31/385; A61K 31/355; A61K 31/22

(52) U.S. Cl. 514/52; 514/251; 514/547; 514/440; 514/276; 514/350; 514/356; 514/458; 514/474

(57) ABSTRACT

The invention describes a novel combination of neutraceuticals. The invention also provides novel kits comprising a novel combination of neutraceuticals. The invention also provides methods for treating symptoms or delaying the progression of neuropathy, e.g., diabetic neuropathy, ischemic neuropathy, metabolic neuropathy, neuropathy due to aging, HIV neuropathy, chemotherapeutic-induced neuropathy, para-neoplastic neuropathy, metabolic neuropathy, and the like.

### RELATED APPLICATIONS

This application claims the benefit to U.S. Provisional Patent Application No. 60/680,521, filed May 13, 2005.

### FIELD OF THE INVENTION

The invention describes novel combinations of neutraceuticals to treat neuropathy.

### BACKGROUND OF THE INVENTION

Nueropathy is, in its broadest terms, is a dysfunction of the nervous system. It can exist as sensory neuropathy in which the peripheral sensory system is dysfunctional, autonomic neuropathy where there are disorders of blood pressure regulation and other functions of the autonomic nervous system and motor neuropathy. Sensory neuropathy and autonomic neuropathy occur in patients who are diabetic. The exact etiology of neuropathy is complex and, in diabetics, is thought to be related to high blood glucose, microvascular disease, increased polyol (sorbitol/aldose reductase) pathway flux, production of advanced glycation end-products, generation of reactive oxygen species₁, and activation of diacylglycerol and protein kinase C isoforms and impaired neuronal repair mechanisms. This increased understanding of the underlying disease mechanism yields a wide variety of new potential therapeutic targets. Besides impaired sensation and associated pain, diabetic neuropathy can lead to serious complications such as diabetic foot ulcers and associated infections. It is the late complications of neuropathy that result in a significant increase in healthcare utilization.

The current mainstay of treatment for diabetic neuropathy is strict control of blood sugar in an attempt to prevent diabetes related complications such as neuropathy. A number of treatment strategies are available for controlling painful neuropathy including topical agents, tricyclic antidepressants, gabapentin and well as a variety of combination therapies. Secondary treatment includes management of patients by a diabetes team, which includes foot care to prevent diabetic ulcers. There is currently no treatment in the U.S. aimed directly at the treatment of neuropathy. There is a consensus based on animal models that early detection and initiation of treatment hold the greatest promise for combating neuropathy. The invention is directed to these, as well as other, important ends.

### SUMMARY OF THE INVENTION

The invention describes novel combinations of neutraceuticals for treating and/or preventing neuropathy, such as diabetic neuropathy or symptomatic diabetic peripheral neuropathy. It has been discovered that the combination of neutraceuticals described herein provides unexpected beneficial results in the treatment of neuropathy and/or the treatment of one or more symptoms associated with neuropathy.

Accordingly, the present invention provides a nutraceutical formulation for treating or preventing neuropathy, the formulation comprising alpha lipoic acid, at least one GLA compound, at least one vitamin C compound, and at least one vitamin B compound.

One embodiment of the present invention is the nutraceutical formulation wherein the alpha lipoic acid is either a racemic mixture or a D enantiomer. Another embodiment of the present invention is the nutraceutical formulation wherein at least one GLA compound is selected from the group comprising borage oil, ascorbylated borage oil, evening primrose oil, blackcurrant, fungal oil, hemp oil, and a combination thereof.

One example of the present invention is the nutraceutical formulation wherein at least one vitamin C compound is vitamin C complex. Another example of the present invention is the nutraceutical formulation wherein at least one vitamin B compound comprises at least one of thiamine and benfotiamine.

In one embodiment of the present invention, the neutraceutical formulation further comprises at least one of fish oil, vitamin B6, vitamin B12, a vitamin B6/B12, and vitamin E, wherein fish oil comprises at least one of omega-3 essential fatty acids and omega-6 essential fatty acids.

In one embodiment, the neutraceutical formulation of the present invention is in an oral liquid dosage form. In another embodiment, the neutraceutical formulation of the present invention is in an oral solid dosage form. In yet another embodiment, the neutraceutical formulation of the present invention is in the form of at least first and second oral solid dosage forms. In one embodiment, the neutraceutical formulation further comprises, in each of the first and second solid oral dosage forms, at least one pharmaceutically acceptable excipient.

The present invention is also directed to a nutraceutical formulation for treating diabetic neuropathy, the formulation comprising an alpha lipoic acid, at least one GLA compound, at least one vitamin C compound, at least one vitamin B compound, and, optionally, acceptable amounts of additives selected from the group comprising at least one of fish oil, vitamin B6, vitamin B12, a vitamin B6/B12 combination, and vitamin E. One embodiment of the present invention is the nutraceutical formulation wherein the GLA is selected from the group consisting of borage oil, ascorbylated borage oil, evening primrose oil, blackcurrant, fungal oil, hemp oil, and mixtures thereof.

One example of the present invention is the nutraceutical formulation wherein at least one vitamin C compound is vitamin C complex. Another example of the present invention is the nutraceutical formulation wherein at least one vitamin B compound comprises thiamine, benfotiamine, and mixtures thereof.

In one embodiment, the neutraceutical formulation of the present invention further comprises at least one of fish oil, vitamin B6, vitamin B12, a vitamin B6/B12, and vitamin E.

The present invention is also directed to a nutraceutical formulation for treating symptomatic diabetic peripheral neuropathy, the formulation comprising an alpha lipoic acid, at least one GLA compound, at least one vitamin C compound, at least one vitamin B compound, and, optionally, acceptable amounts of additives selected from the group comprising at least one of fish oil, vitamin B6, vitamin B12, a vitamin B6/B12 combination, and vitamin E.

The present invention also provides a nutraceutical formulation for treating neuropathy, the formulation for daily intake comprising alpha lipoic acid, borage oil, vitamin C, and benfotiamine. One example of the present invention is the nutraceutical formulation wherein the alpha lipoic acid is in an amount from about 120 mg/day to about 2400 mg/day. Another example of the present invention is the nutraceutical formulation wherein the borage oil is in an amount from about 2000 mg/day to about 6000 mg/day. Yet another example of the present invention is the nutraceutical formulation wherein the vitamin C is in an amount from about 100 mg/day to about 900 mg/day. Some embodiments of the present invention is the nutraceutical formulation wherein the benfotiamine is in an amount from about 25 mg/day to about 400 mg/day.

One aspect of the present invention is the nutraceutical formulation wherein the formulation for daily intake comprises alpha lipoic acid in an amount of about 1200 mg/day, borage oil in an amount of about 3900 mg/day, vitamin C in an amount of about 500 mg/day, and benfotiamine in an amount of about 150 mg/day.

The present invention is further directed to a method for treating neuropathy comprising the step of administering an effective amount of a nutraceutical formulation comprising an alpha lipoic acid, at least one GLA compound, at least one vitamin C compound, at least one vitamin B compound. In one or more embodiments of the present invention, the nutraceutical formulation further comprises at least one of fish oil, vitamin B6, vitamin B12, a vitamin B6/B12 combination, and vitamin E.

### DETAILED DESCRIPTION OF THE INVENTION

As used throughout the disclosure, the following terms, unless otherwise indicated, shall be understood to have the following meanings.

“Nutraceutical” refers to and includes any compound that provides medicinal or health benefits, including the prevention and treatment of disease. A nutraceutical may be a naturally nutrient-rich or medicinally active food, or it may be a specific component of a food, such as vitamins and minerals. The neutraceuticals may be in the form of pharmaceutically acceptable salts; may be in the form of stereoisomers and/or enantiomers; may be in the form of pro-drugs; and/or may be in the form of derivatives thereof. The neutraceuticals of the invention are commercially available or can be made by methods known in the art.

“Antioxidant” refers to and includes any compound that can react and quench a free radical.

“Alpha lipoic acid compound” refers to an antioxidant that serves as a coenzyme in the Krebs cycle and in the production of cellular energy. It is also refered to as lipoic acid or thioctic acid, and is mainly derived from dietary sources, such as spinach, liver, brewer's yeast.

“Borage oil” refers to the oil derived from the seeds of the borage plant (Borago officinalis), a member of the Boraginaceae family. Borage oil, also known as starflower oil and borage seed oil, is a rich source of the long-chain polyunsaturated fatty acid gamma-linolenic acid (GLA). The health benefits of borage oil may be attributed to GLA. GLA is an unusual constituent of living matter and is found in very few plants. These include, in addition to borage, evening primrose, blackcurrant and hemp. The amount of GLA in borage oil, as the percentage of total fatty acid content, ranges from about 20% to 30%. Typical borage oil supplements contain approximately 24% GLA.

“GLA” refers to and includes any compound that is an all cis n-6 long-chain polyunsaturated fatty acid. It is comprised of 18 carbon atoms and three double bonds. GLA is also known as GLA; 18: 3n-6 and gamolenic acid. Chemically, it is known as 6, 9, 12-octadecatrienoic acid; (Z, Z, Z)-6, 9, 12-octadecatrienoic acid, and cis-6, cis-9, cis-12-octadecatrienoic acid. GLA is present in borage oil in the form of triglycerides. GLA is concentrated in the sn-2 position in the triglycerides.

“Benfotiamine” refers to S-benzoylthiamine-O-monophosphate and is a synthetic fat-soluble form of thiamine (vitamin B1). It is an effective anti-oxidant and ancillary effects include protection from advanced glycation end products (AGEs) that can affect nerves and vascular cells in diabetes. It is also highly effective at increasing plasma and cellular levels of thiamine diphosphate (TDP), a metabolically active coenzyme form of B1.

“Therapeutic agent” includes any therapeutic agent that can be used to treat or prevent the diseases described herein. “Therapeutic agents” include, for example, analgesics, anticonvulsants, antidepressants, local anesthetics, and antiarrhythmics, and topical treatment with capsaicin cream, and the like. Therapeutic agent includes the pro-drugs and pharmaceutical derivatives thereof including, but not limited to, the corresponding vitamin suplements.

“Patient” refers to animals, preferably mammals, most preferably humans, and includes males and females, and children and adults.

“Therapeutically effective amount” refers to the amount of the compound and/or composition that is effective to achieve its intended purpose.

The invention provides safe and effective methods of treating and/or delaying the progression of neuropathy in a patient in need thereof by administering a therapeutically effective amount of the neutraceuticals of the invention. “Neuropathy” refers to a disturbance in the function of a nerve or particular group of nerves. The nerves affected are outside the brain and spinal cord and are known as peripheral nerves. Thus, neuropathy is often referred to as peripheral neuropathy. Typical symptoms of neuropathy may include numbness, tingling, or pain in the toes, feet, legs, hands, arms, and fingers, wasting of the muscles of the feet or hands, indigestion, nausea, or vomiting, diarrhea or constipation, dizziness or faintness due to a drop in postural blood pressure, problems with urination, erectile dysfunction (impotence) or vaginal dryness, weakness, weight loss, and depression. The neutraceuticals of the invention may be administered separately or may be administered in the form of one or more compositions that further comprise a carrier. The neutraceuticals of the invention may be administered daily to the patient.

The invention provides safe and effective methods of treating and/or delaying the progression of diabetic neuropathy in a patient in need thereof by administering a therapeutically effective amount of the neutraceuticals of the invention. The invention also provides safe and effective methods for ameliorating the neurovascular dysfunction of diabetic neuropathy in a patient in need thereof by administering a therapeutically effective amount of the neutraceuticals of the invention. “Diabetic neuropathy” refers to a nerve disorder(s) caused by or associated with diabetes, characterized by symptoms including numbness and sometimes pain and weakness in the hands, arms, feet, and/or legs. Problems may also occur in every organ system, including the digestive tract, heart, and sex organs. The “diabetic neuropathy” may be symptomatic diabetic peripheral neuropathy. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of ischemic neuropathy in a patient in need thereof by administering a therapeutically effective amount of the neutraceuticals of the invention. “Ischemic neuropathy” refers to endoneurial ischemia developed because of increased endoneurial vascular resistance to hyperglycemic blood and due to various disorders, such as peripheral vascular occlusive diseases, necrotizing vasculitides, diabetes mellitus and nerve compression or trauma. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delayng the progression of metabolic syndrome and/or metabolic neuropathy in a patient in need thereof by administering a therapeutically effective amount of at the neutraceuticals of the invention. “Metabolic neuropathy” refers to a disease of the nerves caused by a problem regulating chemical processes in the body. In some cases, nerve damage is caused by the inability to properly use energy in the body. In other cases, various toxins accumulate and damage nerves. Some metabolic disorders are inherited, while others are acquired during life through various diseases. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of neuropathy due to aging in a patient in need thereof by administering a therapeutically effective amount of the neutraceuticals of the invention. “Neuropathy due to aging” refers to diabetic neuropathy due to advanced age and increased duration of diabetes. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of HIV neuropathy in a patient in need thereof by administering a therapeutically effective amount of the neutraceuticals of the invention. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of chemotherapeutic-induced neuropathy in a patient in need thereof by administering a therapeutically effective amount of the neutraceuticals of the invention. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of para-neoplastic neuropathy in a patient in need thereof by administering a therapeutically effective amount of the neutraceuticals of the invention. The neutraceuticals of the invention may be administered separately or may be administered in the form of one or more compositions that further comprise a carrier. The neutraceuticals of the invention may be administered daily to the patient.

In another embodiment, the invention provides safe and effective methods of treating and/or delaying the progression of multiple sclerosis and/or neuropathy associated with multiple sclerosis in a patient in need thereof by administering a therapeutically effective amount of the neutraceuticals of the invention. The neutraceuticals of the invention may be administered separately or may be administered in the form of one or more compositions that further comprise a carrier. The neutraceuticals of the invention may be administered daily to the patient.

In one embodiment, the “neutraceuticals of the invention” comprise (i) an alpha lipoic acid compound, pro-drug, metabolite or derivative thereof; (ii) GLA and/or a GLA-contaning compound, pro-drug, metabolite or derivative thereof; (iii) a vitamin C compound, pro-drug, metabolite or derivative thereof; and (iv) a vitamin B compound, pro-drug, metabolite or derivative thereof.

In one embodiment, the “neutraceuticals of the invention” comprise comprise alpha lipoic acid (e.g., as a racemic mixture, as the D enantiomer); one or more compounds that comprise GLA (e.g., borage oil, ascorbylated borage oil, evening primrose oil, blackcurrant, fungal oil, hemp oil); one or more vitamin C compounds (e.g., vitamin C, vitamin C complex); and one or more vitamin B1 compounds (e.g., thiamine, benfotiamine). The neutraceuticals may be administered individually or may be administered in the form of one, two or three compositions.

The neutraceuticals of the invention may further optionally comprise fish oil (e.g., omega-3 essential fatty acids and/or omega-6 essential fatty acids), vitamin B6, vitamin B12, a vitamin B6/B12 combination, vitamin E, or a mixture of two or more thereof. In another embodiment, the neutraceuticals of the invention may further optionally comprise fish oil (e.g., omega-3 essential fatty acids and/or omega-6 essential fatty acids), a vitamin B6/B12 combination, or a mixture thereof.

In one embodiment, the invention provides neutraceuticals for daily intake that comprise alpha lipoic acid in an amount from about 120 mg/day to about 2400 mg/day, borage oil in an amount from about 2000 mg/day to about 6000 mg/day, vitamin C in an amount from about 100 mg/day to about 900 mg/day, and benfotiamine in an amount from about 25 mg/day to about 400 mg/day. In another embodiment, the invention provides neutraceuticals for daily intake comprising alpha lipoic acid in an amount from about 900 mg/day to about 2000 mg/day, borage oil in an amount from about 3000 mg/day to about 5000 mg/day, vitamin C in an amount from about 300 mg/day to about 700 mg/day, and benfotiamine in an amount from about 75 mg/day to about 225 mg/day. In yet another embodiment of the invention, the neutraceuticals for daily intake comprises alpha lipoic acid in an amount from about 1100 mg/day to about 1300 mg/day, borage oil in an amount from about 3700 mg/day to about 4000 mg/day, vitamin C in an amount from about 450 mg/day to about 550 mg/day, and benfotiamine in an amount from about 125 mg/day to about 175 mg/day. In yet another embodiment, the neutraceuticals for daily intake comprises alpha lipoic acid in an amount of about 1200 mg/day, borage oil in an amount of about 3900 mg/day, vitamin C in an amount of about 500 mg/day, and benfotiamine in an amount of about 150 mg/day. It has been unexpectedly discovered that the combination of neutraceuticals of the invention may provide synergistic (i.e., greater than additive) effects in the treatment of neuropathy.

The invention is also based on the discovery that neutraceuticals of the invention may be used in conjunction with other therapeutic agents for co-therapies, partially or completely, in place of other therapeutic agents, such as, for example, analgesics, anticonvulsants, antidepressants, local anesthetics, and antiarrhythmics, and topical treatment with capsaicin cream.

The neutraceuticals (e.g., compounds and/or compositions) of the invention can be administered by any available and effective delivery system including, but not limited to, orally, bucally, parenterally, by inhalation spray, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques. Different neutraceuticals may be administered by different delivery systems and/or by different dosage forms (e.g., vitamin C may be orally administered in the form of a tablet and fish oil may be orally administered in the form of a liquid).

Solid dosage forms for oral administration can include capsules, sustained-release capsules, tablets, sustained release tablets, chewable tablets, sublingual tablets, effervescent tablets, pills, powders, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for rectal administration of the compounds and compositions of the invention can be prepared by mixing the compounds or compositions with a suitable nonirritating excipient such as cocoa butter and polyethylene glycols which are solid at room temperature but liquid at rectal temperature, such that they will melt in the rectum and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compositions of this invention can further include conventional excipients, i.e., organic or inorganic carrier substances suitable for parenteral application which do not deleteriously react with the active compounds. Suitable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances, which increase the viscosity of the suspension and include, for example, sodium carboxymethyl cellulose, sorbitol and/or dextran. Optionally, the suspension may also contain stabilizers.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailabilty of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a vitamin supplement dispersed therein or may comprise the vitamin supplement in pure, preferably crystalline, solid form. For sustained release administration, microparticle dosage forms comprising pure, preferably crystalline, vitamin supplements are preferred. The therapeutic dosage forms of this aspect of the invention may be of any configuration suitable for sustained release.

Nanoparticle sustained release dosage forms are preferably biodegradable and, optionally, bind to the vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Preferred larger microparticle dosage forms of the invention release the vitamin supplements for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release dosage form is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Particular sustained release dosage forms of the invention comprise biodegradable microparticles or nanoparticles. More particularly, biodegradable microparticles or nanoparticles are formed of a polymer containing matrix that biodegrades by random, nonenzymatic, hydrolytic scissioning to release vitamin supplements, thereby forming pores within the particulate structure.

The compounds and compositions of the invention can be formulated as salt forms. Salts include, for example, alkali metal salts and addition salts of free acids or free bases. The nature of the salt is not critical. Suitable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

Transdermal compound administration, which is known to one skilled in the art, involves the delivery of compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

The compositions can also be applied topically using a transdermal system, such as one of an acrylic-based polymer adhesive with a resinous crosslinking agent impregnated with the composition and laminated to an impermeable backing. In a particular embodiment, the compositions of the invention are administered as a transdermal patch, more particularly as a sustained-release transdermal patch. The transdermal patches of the invention can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material. Suitable transdermal patches are described in more detail in, for example, U.S. Pat. Nos. 5,262,165, 5,948,433, 6,010,715 and 6,071,531, the disclosures of each of which are incorporated by reference herein in their entireties as though set forth in full.

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The invention also provides neutraceutical kits comprising one or more containers filled with one or more of the ingredients of the compounds and/or compositions of the invention, including, at least, one or more of the novel vitamin supplements described herein. Associated with such kits can be additional therapeutic agents or compositions (e.g., analgesics, anticonvulsants, antidepressants, local anesthetics, and antiarrhythmics, and topical treatment with capsaicin cream, and the like, and mixtures of two or more thereof), devices for administering the compositions, and notices in the form prescribed by a governmental agency regulating the manufacture, use or sale of neutraceuticals and/or pharmaceuticals which reflects any necessary approval by the agency of manufacture, use or sale for humans.

### EXAMPLES

The following non-limiting examples further describe and enable one of ordinary skill in the art to make and use the invention.

### Example 1

In this example, the following combination of neutraceuticals was assembled and administered to human patients for the alleviation of the symptoms associated with diabetic neuropathy:

| Supplement | Number of pills/day | Prescribed Dose |
|---|---|---|
| Borage Oil | 4 | 3900 (mg/day) |
| Alpha Lipoic Acid | 2 | 600 (mg/day) |
| Vitamin C | 1 | 500 (mg/day) |
| Vitamin E | 1 | 400 (IU/day) |

### Example 2

In this example, the following combination of neutraceuticals was assembled and administered to human patients for the alleviation of the symptoms associated with diabetic neuropathy:

| Supplement | Number of pills/day | Prescribed Dose |
|---|---|---|
| Borage Oil | 4 | 3900 (mg/day) |
| Alpha Lipoic Acid | 4 | 1200 (mg/day) |
| Vitamin C | 1 | 500 (mg/day) |
| Vitamin E | 1 | 400 (IU/day) |

### Example 3

In this example, the following combination of neutraceuticals was assembled and administered to human patients for the alleviation of the symptoms associated with diabetic neuropathy:

| Supplement | Number of pills/day | Prescribed Dose |
|---|---|---|
| Borage Oil | 4 | 3900 (mg/day) |
| Alpha Lipoic Acid | 4 | 1200 (mg/day) |
| Vitamin C | 1 | 500 (mg/day) |
| Benfotiamine | 1 | 150 (mg/day) |

### Example 4

In this example, the following combination of neutraceuticals is assembled and administered to human patients for the alleviation of the symptoms associated with diabetic neuropathy:

| Supplement | Number of pills/day | Prescribed Dose |
|---|---|---|
| Borage Oil | 4 | 3900 (mg/day) |
| Alpha Lipoic Acid | 4 | 1200 (mg/day) |
| Vitamin C | 1 | 500 (mg/day) |
| Thiamine | 1 | 150 (mg/day) |

### Example 5

In this example, the following combination of neutraceuticals is assembled and administered to human patients for the alleviation of the symptoms associated with diabetic neuropathy:

| Supplement | Number of pills/day | Prescribed Dose |
|---|---|---|
| Borage Oil | 4 | 3900 (mg/day) |
| Alpha Lipoic Acid | 4 | 1200 (mg/day) |
| Vitamin C | 1 | 500 (mg/day) |
| Benfotiamine | 1 | 150 (mg/day) |
| Vitamin B6/B12 | 1 | 1-200 (μg/day) |

Although the invention has been set forth in detail, one skilled in the art will appreciate that numerous changes and modifications can be made to the invention, and that such changes and modifications can be made without departing from the spirit and scope of the invention.

We claim:

1. A nutraceutical formulation for treating or preventing neuropathy, the formulation comprising alpha lipoic acid, at least one GLA compound, at least one vitamin C compound, and at least one vitamin B compound.

2. The nutraceutical formulation of claim 1, wherein the alpha lipoic acid is a racemic mixture.

3. The nutraceutical formulation of claim 1, wherein the alpha lipoic acid is a D enantiomer.

4. The nutraceutical formulation of claim 1, wherein said at least one GLA compound is selected from the group comprising borage oil, ascorbylated borage oil, evening primrose oil, blackcurrant, fungal oil, hemp oil, and a combination thereof.

5. The nutraceutical formulation of claim 1, wherein said at least one vitamin C compound is vitamin C complex.

6. The nutraceutical formulation of claim 1, wherein said at least one vitamin B compound comprises at least one of thiamine and benfotiamine.

7. The neutraceutical formulation of claim 1, further comprising at least one of fish oil, vitamin B6, vitamin B12, a vitamin B6/B12, and vitamin E.

8. The neutraceutical formulation of claim 7, wherein fish oil comprises at least one of omega-3 essential fatty acids and omega-6 essential fatty acids.

9. The neutraceutical formulation of claim 1 in an oral liquid dosage form.

10. The neutraceutical formulation of claim 1 in an oral solid dosage form.

11. The neutraceutical formulation of claim 1 in the form of at least first and second oral solid dosage forms.

12. The neutraceutical formulation of claim 1 further comprising, in each of the first and second solid oral dosage forms, at least one pharmaceutically acceptable excipient.

13. A nutraceutical formulation for treating diabetic neuropathy, the formulation comprising an alpha lipoic acid, at least one GLA compound, at least one vitamin C compound, at least one vitamin B compound, and, optionally, acceptable amounts of additives selected from the group comprising at least one of fish oil, vitamin B6, vitamin B12, a vitamin B6/B12 combination, and vitamin E.

14. The nutraceutical formulation of claim 13, wherein the GLA is selected from the group consisting of borage oil, ascorbylated borage oil, evening primrose oil, blackcurrant, fungal oil, hemp oil, and mixtures thereof.

15. The nutraceutical formulation of claim 13, wherein said at least one vitamin C compound is vitamin C complex.

16. The nutraceutical formulation of claim 13, wherein said at least one vitamin B compound comprises thiamine, benfotiamine, and mixtures thereof.

17. The neutraceutical formulation of claim 13, further comprising at least one of fish oil, vitamin B6, vitamin B12, a vitamin B6/B12, and vitamin E.

18. A nutraceutical formulation for treating symptomatic diabetic peripheral neuropathy, the formulation comprising an alpha lipoic acid, at least one GLA compound, at least one vitamin C compound, at least one vitamin B compound, and, optionally, acceptable amounts of additives selected from the group comprising at least one of fish oil, vitamin B6, vitamin B12, a vitamin B6/B12 combination, and vitamin E.

19. A nutraceutical formulation for treating neuropathy, the formulation for daily intake comprising alpha lipoic acid, borage oil, vitamin C, and benfotiamine.

20. The nutraceutical formulation of claim 19, wherein the alpha lipoic acid is in an amount from about 120 mg/day to about 2400 mg/day.

21. The nutraceutical formulation of claim 19, wherein the borage oil is in an amount from about 2000 mg/day to about 6000 mg/day.

22. The nutraceutical formulation of claim 19, wherein the vitamin C is in an amount from about 100 mg/day to about 900 mg/day.

23. The nutraceutical formulation of claim 19, wherein the benfotiamine is in an amount from about 25 mg/day to about 400 mg/day.

24. The nutraceutical formulation of claim 19, wherein the formulation for daily intake comprises alpha lipoic acid in an amount of about 1200 mg/day, borage oil in an amount of about 3900 mg/day, vitamin C in an amount of about 500 mg/day, and benfotiamine in an amount of about 150 mg/day.

25. A method for treating neuropathy comprising the step of administering an effective amount of a nutraceutical formulation comprising an alpha lipoic acid, at least one GLA compound, at least one vitamin C compound, at least one vitamin B compound.

26. The method of claim 25 wherein the nutraceutical formulation further comprises at least one of fish oil, vitamin B6, vitamin B12, a vitamin B6/B12 combination, and vitamin E.

27. The method of claim 25 wherein the nutraceutical composition is in an oral liquid dosage form.

28. The method of claim 25 wherein the nutraceutical composition is in an oral solid dosage form.

29. The method of claim 25, wherein the neuropathy is diabetic neuropathy.

30. The method of claim 25, wherein the neuropathy is symptomatic diabetic peripheral neuropathy.
